(19) United States
(12) Patent Application Publication
(10) Pub. No.: US 2019/0282205 A1
(43) Pub. Date: Sep. 19, 2019

(54) ULTRASOUND IMAGING SYSTEM AND ULTRASOUND IMAGING METHOD

(71) Applicant: QISDA CORPORATION, Taoyuan City (TW)

(72) Inventor: Yu-Teng Tung, Hsinchu City (TW)

(21) Appl. No.: 16/219,761

(22) Filed: Dec. 13, 2018

(30) Foreign Application Priority Data
Mar. 19, 2018 (TW) 107109193

(51) Int. Cl. A61B 8/00; G06N 3/08; A61B 8/08; G02B 26/10; H04N 19/117; G06T 5/00

(52) U.S. Cl. CPC A61B 8/4461; G06N 3/08; G06T 5/002; G02B 26/10; H04N 19/117; A61B 8/5238

(57) ABSTRACT

An ultrasound imaging system includes an ultrasound probe, a filter, a first neural network and a processor. The ultrasound probe generates a target ultrasound image and a plurality of first reference ultrasound images by a plurality of first scanning parameters. The filter filters the target ultrasound image to generate a first filtered ultrasound image. The first neural network filters the target ultrasound image according to the first reference ultrasound images to generate a second filtered ultrasound image. The processor combines the first filtered ultrasound image and the second filtered ultrasound image to form a compound ultrasound image.

### BACKGROUND OF THE INVENTION

### 1. Field of the Invention

The invention relates to an ultrasound imaging system and an ultrasound imaging method and, more particularly, to an ultrasound imaging system and an ultrasound imaging method capable of reducing noise effectively.

### 2. Description of the Prior Art

Since ultrasound scanning equipment does not destroy material structure and cell, the ultrasound scanning equipment is in widespread use for the field of material and clinical diagnosis. In general, an ultrasound image may have some noise therein, wherein the speckle noise is the most complicated to be processed. The speckle noise is generated by coherent interference while the ultrasound is scattered. The speckle noise will reduce resolution of the ultrasound image, such that the accuracy of the ultrasound image is affected. Therefore, how to reduce noise effectively for ultrasound scanning technology has become a significant research issue.

### SUMMARY OF THE INVENTION

An objective of the invention is to provide an ultrasound imaging system and an ultrasound imaging method capable of reducing noise effectively, so as to solve the aforesaid problems.

According to an embodiment of the invention, an ultrasound imaging system comprises an ultrasound probe, a filter, a first neural network and a processor. The ultrasound probe generates a target ultrasound image and a plurality of first reference ultrasound images by a plurality of first scanning parameters. The filter filters the target ultrasound image to generate a first filtered ultrasound image. The first neural network filters the target ultrasound image according to the first reference ultrasound images to generate a second filtered ultrasound image. The processor combines the first filtered ultrasound image and the second filtered ultrasound image to form a compound ultrasound image.

According to another embodiment of the invention, an ultrasound imaging method comprises steps of generating a target ultrasound image and a plurality of first reference ultrasound images by a plurality of first scanning parameters; filtering the target ultrasound image by a filter to generate a first filtered ultrasound image; filtering the target ultrasound image by a first neural network according to the first reference ultrasound images to generate a second filtered ultrasound image; and combining the first filtered ultrasound image and the second filtered ultrasound image to form a compound ultrasound image.

As mentioned in the above, after generating the target ultrasound image and the reference ultrasound images by different scanning parameters, the invention filters the target ultrasound image by the filter and filters the target ultrasound image by the neural network according to the reference ultrasound images, so as to generate the filtered ultrasound images. Then, the invention combines the filtered ultrasound images to form the compound ultrasound image. Since the filter has filtered noise from the filtered ultrasound image and the neural network has filtered noise from the filtered ultrasound image, the invention can reduce noise of the compound ultrasound image effectively, so as to improve the accuracy of the compound ultrasound image.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a functional block diagram illustrating an ultrasound imaging system according to an embodiment of the invention.

FIG. 2 is a flowchart illustrating an ultrasound imaging method according to an embodiment of the invention.

FIG. 3 is a functional block diagram illustrating an ultrasound imaging system according to another embodiment of the invention.

FIG. 4 is a functional block diagram illustrating an ultrasound imaging system according to another embodiment of the invention.

FIG. 5 is a flowchart illustrating an ultrasound imaging method according to another embodiment of the invention.

FIG. 6 is a functional block diagram illustrating an ultrasound imaging system according to another embodiment of the invention.

### DETAILED DESCRIPTION

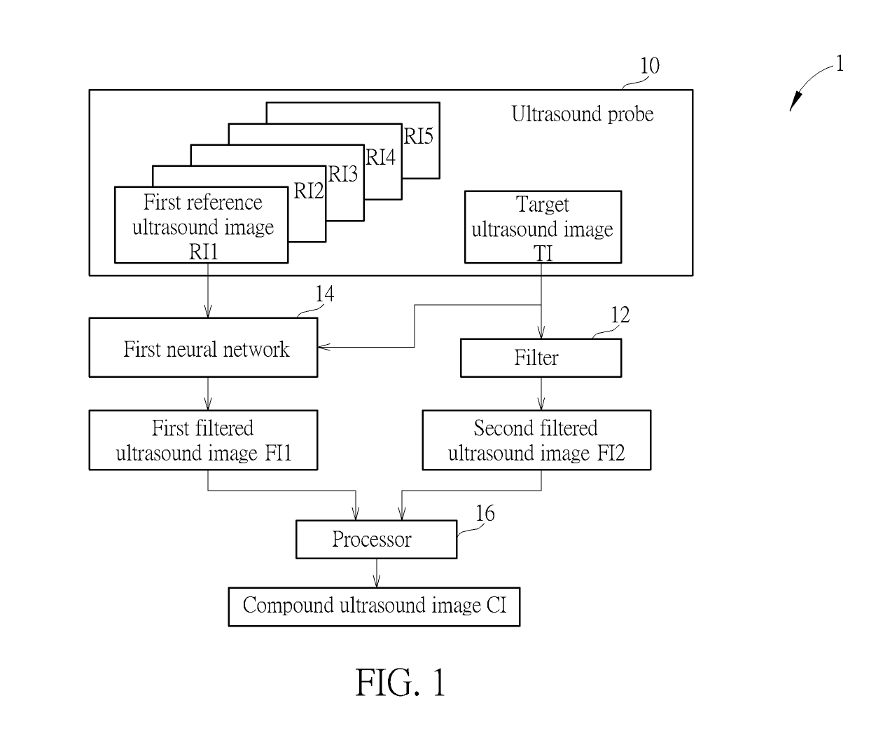

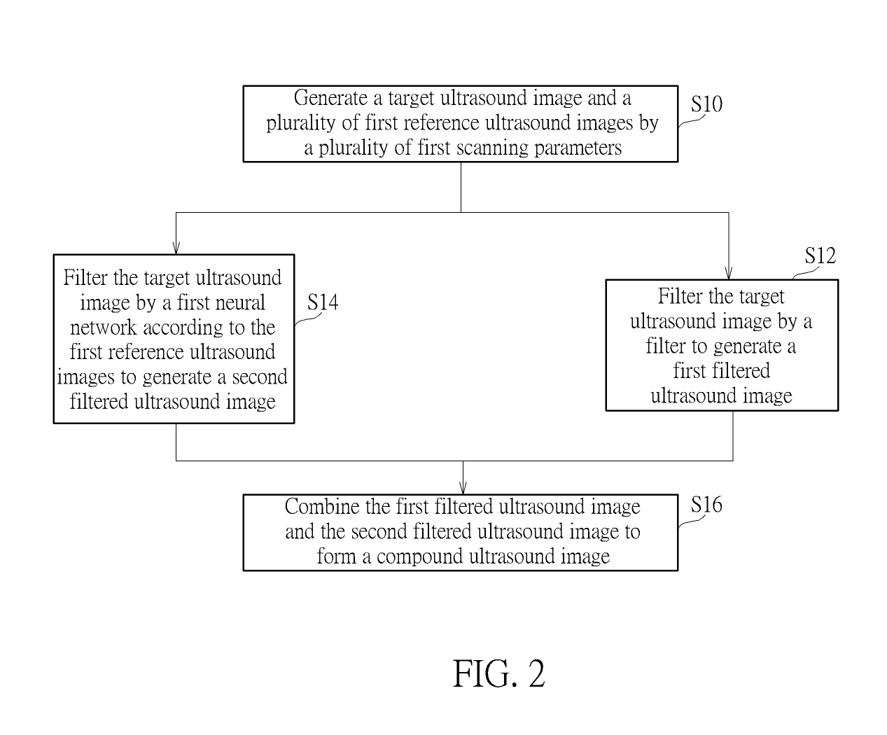

Referring to FIGS. 1 and 2, FIG. 1 is a functional block diagram illustrating an ultrasound imaging system 1 according to an embodiment of the invention and FIG. 2 is a flowchart illustrating an ultrasound imaging method according to an embodiment of the invention. The ultrasound imaging method shown in FIG. 2 can be implemented by the ultrasound imaging system 1 shown in FIG. 1.

As shown in FIG. 1, the ultrasound imaging system 1 comprises an ultrasound probe 10, a filter 12, a first neural network 14 and a processor 16. In this embodiment, the filter 12, the first neural network 14 and the processor 16 may be disposed in a computer (not shown), and the computer may communicate with the ultrasound probe 10 to transmit signals. In another embodiment, the filter 12, the first neural network 14 and the processor 16 may be integrated into the ultrasound probe 10 according to practical applications.

When using the ultrasound imaging system 1 to perform ultrasound scanning for a target object (not shown), an operator may operate the ultrasound probe 10 to emit ultrasound signals to the target object by a plurality of first scanning parameters and receive the ultrasound signals reflected and/or scattered by the target object, so as to generate a target ultrasound image TI and a plurality of first reference ultrasound images RI1-RI5 (step S10 in FIG. 2). In this embodiment, the first scanning parameter may be a scanning frequency or a scanning angle. For example, if the first scanning parameter is the scanning frequency, the operator may operate the ultrasound probe 10 to emit ultrasound signals to the target object by six different scanning frequencies and then receive the ultrasound signals reflected and/or scattered by the target object, so as to generate one target ultrasound image TI and five first reference ultrasound images RI1-RI5, wherein the scanning angles of the target ultrasound image TI and the first reference ultrasound images RI1-RI5 may be constant. Furthermore, if the first scanning parameter is the scanning angle, the operator may operate the ultrasound probe 10 to emit ultrasound signals to the target object by six different scanning angles and then receive the ultrasound signals reflected and/or scattered by the target object, so as to generate one target ultrasound image TI and five first reference ultrasound images RI1-RI5, wherein the scanning frequencies of the target ultrasound image TI and the first reference ultrasound images RI1-RI5 may be constant. It should be noted that the invention may determine to use how many first scanning parameters to generate how many first reference ultrasound images according to practical applications. That is to say, the number of the first reference ultrasound images is not limited to five.

After generating the target ultrasound image TI, the target ultrasound image TI is inputted into the filter 12, such that the filter 12 filters the target ultrasound image TI to generate a first filtered ultrasound image FI1 (step S12 in FIG. 2). In this embodiment, the filter 12 may be a mean filter, a median filter, a Gaussian filter, a bilateral filter, a guided filter, a Wiener filter, an adaptive median filter, or other filters or algorithms capable of filtering noise.

Furthermore, after generating the target ultrasound image TI and the first reference ultrasound images RI1-RI5, the target ultrasound image TI and the first reference ultrasound images RI1-RI5 are inputted into the first neural network 14, such that the first neural network 14 filters the target ultrasound image TI according to the first reference ultrasound images RI1-RI5 to generate a second filtered ultrasound image FI2 (step S14 in FIG. 2). In this embodiment, the first neural network 14 may be a convolution neural network (CNN) or the like. In this embodiment, the first neural network 14 has been trained for filtering noise from the target ultrasound image TI. The invention may prepare a plurality of training samples in advance, wherein each of the training samples comprises the aforesaid target ultrasound image TI and the aforesaid first reference ultrasound images RI1-RI5, and the position of the noise in the target ultrasound image TI has been known. Then, the training samples are inputted into the first neural network 14 to train the first neural network 14 to filter the noise from the target ultrasound image TI. It should be noted that the detailed training process of the neural network is well known by one skilled in the art, so it will not be depicted herein in detail.

After generating the first filtered ultrasound image FI1 and the second filtered ultrasound image FI2, the processor 16 combines the first filtered ultrasound image FI1 and the second filtered ultrasound image FI2 to form a compound ultrasound image CI (step S16 in FIG. 2). Since the filter 12 has filtered the noise from the first filtered ultrasound image FI1 and the first neural network 14 has filtered the noise from the second filtered ultrasound image FI2, the invention can reduce the noise of the compound ultrasound image CI effectively, so as to improve the accuracy of the compound ultrasound image CI. In this embodiment, the invention may use some manners including volting/average, neural network, alpha blending, multi-band blending, etc. to combine the first filtered ultrasound image FI1 and the second filtered ultrasound image FI2 to form the compound ultrasound image CI. However, the manner of combining images is not limited to the aforesaid embodiment.

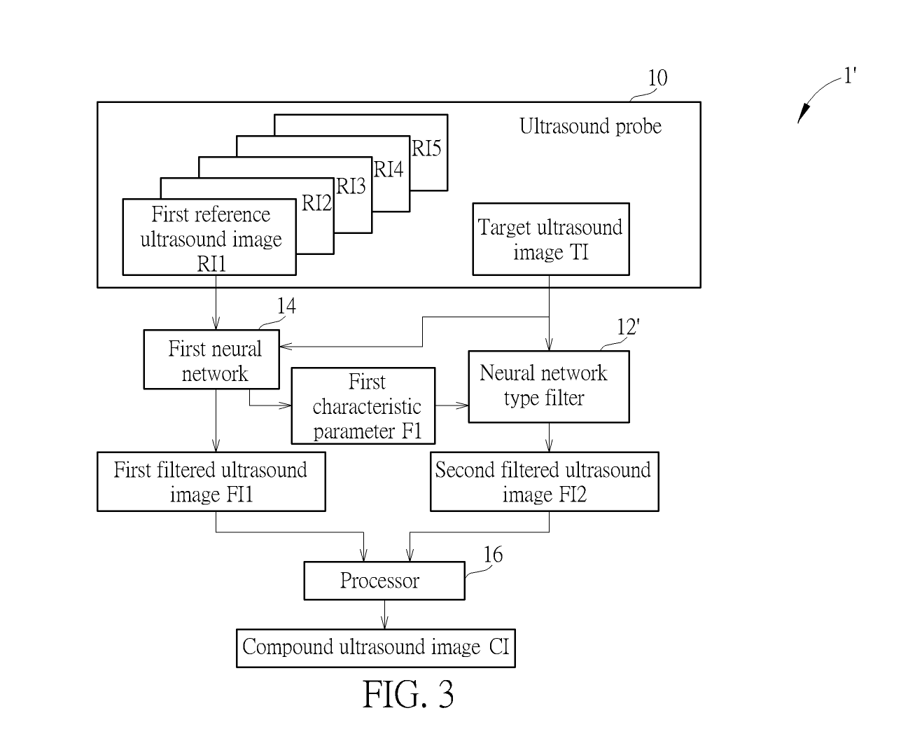

Referring to FIG. 3, FIG. 3 is a functional block diagram illustrating an ultrasound imaging system 1′ according to another embodiment of the invention. The main difference between the ultrasound imaging system 1′ and the aforesaid ultrasound imaging system 1 is that the filter 12′ of the ultrasound imaging system 1′ is a neural network type filter, as shown in FIG. 3. In this embodiment, the filter 12′ may also be a convolution neural network (CNN) or the like. In this embodiment, after generating the target ultrasound image TI and the first reference ultrasound images RI1-RI5, the first neural network 14 filters the target ultrasound image TI first according to the first reference ultrasound images RI1-RI5. Then, the first neural network 14 outputs a first characteristic parameter F1 to the filter 12′ after filtering the target ultrasound image TI. Then, the filter 12′ filters the target ultrasound image TI according to the first characteristic parameter F1 to generate the first filtered ultrasound image FI1. In this embodiment, the neural network type filter 12′ has been trained for filtering noise from the target ultrasound image TI according to the first characteristic parameter F1 generated by the first neural network 14. The invention may prepare a plurality of target ultrasound images TI in advance and the positions of the noises in the target ultrasound images TI have been known. Then, the target ultrasound images TI are inputted into the filter 12′ to train the filter 12′ to filter the noise from the target ultrasound image TI. It should be noted that the detailed training process of the neural network is well known by one skilled in the art, so it will not be depicted herein in detail. Furthermore, the manner of generating the characteristic parameter of the neural network is also well known by one skilled in the art, so it will not be depicted herein in detail. Since the first filtered ultrasound image FI1 is generated according to the first characteristic parameter F1 generated by the first neural network 14, the noise of the compound ultrasound image CI can be further reduced.

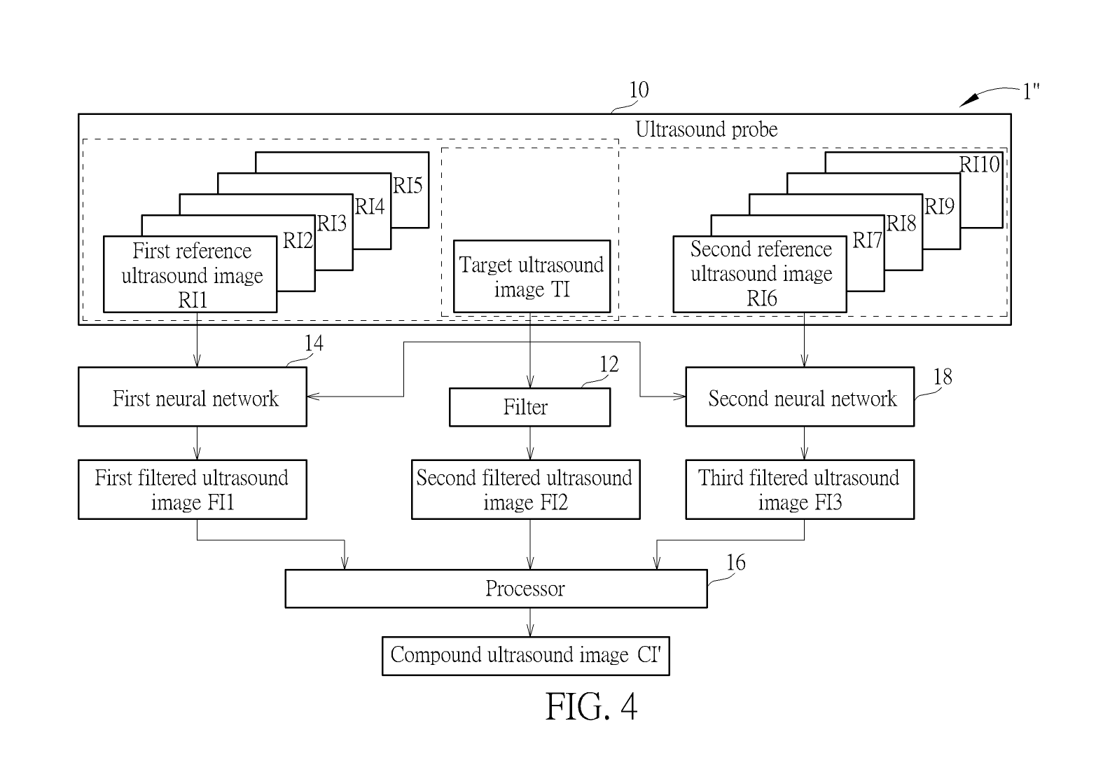

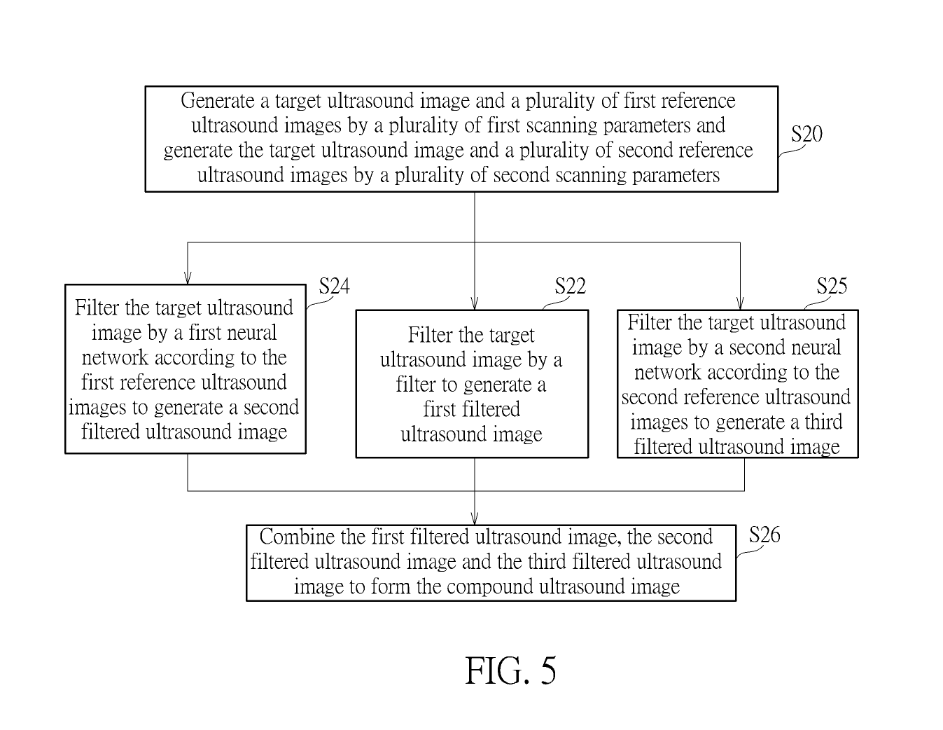

Referring to FIGS. 4 and 5, FIG. 4 is a functional block diagram illustrating an ultrasound imaging system 1″ according to another embodiment of the invention and FIG. 5 is a flowchart illustrating an ultrasound imaging method according to another embodiment of the invention. The ultrasound imaging method shown in FIG. 5 can be implemented by the ultrasound imaging system 1″ shown in FIG. 4. The main difference between the ultrasound imaging system 1″ and the aforesaid ultrasound imaging system 1 is that the ultrasound imaging system 1″ further comprises a second neural network 18, as shown in FIG. 4. In this embodiment, the second neural network 18 may be disposed in the aforesaid computer or integrated into the ultrasound probe 10 according to practical applications. It should be noted that the same elements in FIG. 4 and FIG. 1 are represented by the same numerals, so the repeated explanation will not be depicted herein again.

When using the ultrasound imaging system 1″ to perform ultrasound scanning for a target object (not shown), an operator may operate the ultrasound probe 10 to emit ultrasound signals to the target object by a plurality of first scanning parameters and receive the ultrasound signals reflected and/or scattered by the target object, so as to generate a target ultrasound image TI and a plurality of first reference ultrasound images RI1-RI5 (step S20 in FIG. 5). Furthermore, the operator may operate the ultrasound probe 10 to emit ultrasound signals to the target object by a plurality of second scanning parameters and receive the ultrasound signals reflected and/or scattered by the target object, so as to generate the target ultrasound image TI and a plurality of second reference ultrasound images RI6-RI10 (step S20 in FIG. 5).

In this embodiment, the first scanning parameter may be one of a scanning frequency and a scanning angle, and the second scanning parameter may be another one of the scanning frequency and the scanning angle. For example, if the first scanning parameter is the scanning frequency and the second scanning parameter is the scanning angle, the operator may operate the ultrasound probe 10 to emit ultrasound signals to the target object by six different scanning frequencies and then receive the ultrasound signals reflected and/or scattered by the target object, so as to generate one target ultrasound image TI and five first reference ultrasound images RI1-RI5, wherein the scanning angles of the target ultrasound image TI and the first reference ultrasound images RI1-RI5 may be constant. Then, the operator may operate the ultrasound probe 10 to emit ultrasound signals to the target object by six different scanning angles (including the scanning angle of the target ultrasound image TI generated before) and then receive the ultrasound signals reflected and/or scattered by the target object, so as to generate one target ultrasound image TI and five second reference ultrasound images RI6-RI10, wherein the scanning frequencies of the target ultrasound image TI and the second reference ultrasound images RI6-RI10 may be constant. It should be noted that the invention may determine to use how many first scanning parameters and how many second scanning parameters to generate how many first reference ultrasound images and second reference ultrasound images according to practical applications. That is to say, the number of the first reference ultrasound images and the number of the second reference ultrasound images are not limited to five.

After generating the target ultrasound image TI, the target ultrasound image TI is inputted into the filter 12, such that the filter 12 filters the target ultrasound image TI to generate a first filtered ultrasound image FI1 (step S22 in FIG. 5).

Still further, after generating the target ultrasound image TI and the first reference ultrasound images RI1-RI5, the target ultrasound image TI and the first reference ultrasound images RI1-RI5 are inputted into the first neural network 14, such that the first neural network 14 filters the target ultrasound image TI according to the first reference ultrasound images RI1-RI5 to generate a second filtered ultrasound image FI2 (step S24 in FIG. 5).

Moreover, after generating the target ultrasound image TI and the second reference ultrasound images RI6-RI10, the target ultrasound image TI and the second reference ultrasound images RI6-RI10 are inputted into the second neural network 18, such that the second neural network 18 filters the target ultrasound image TI according to the second reference ultrasound images RI6-RI10 to generate a third filtered ultrasound image FI3 (step S25 in FIG. 5). In this embodiment, the second neural network 18 may be a convolution neural network (CNN) or the like. In this embodiment, the second neural network 18 has been trained for filtering noise from the target ultrasound image TI. The invention may prepare a plurality of training samples in advance, wherein each of the training samples comprises the aforesaid target ultrasound image TI and the aforesaid second reference ultrasound images RI6-RI10, and the position of the noise in the target ultrasound image TI has been known. Then, the training samples are inputted into the second neural network 18 to train the second neural network 18 to filter the noise from the target ultrasound image TI. It should be noted that the detailed training process of the neural network is well known by one skilled in the art, so it will not be depicted herein in detail.

After generating the first filtered ultrasound image FI1, the second filtered ultrasound image FI2 and the third filtered ultrasound image FI3, the processor 16 combines the first filtered ultrasound image FI1, the second filtered ultrasound image FI2 and the third filtered ultrasound image FI3 to form a compound ultrasound image CI′ (step S26 in FIG. 5). Since the filter 12 has filtered the noise from the first filtered ultrasound image FI1, the first neural network 14 has filtered the noise from the second filtered ultrasound image FI2, and the second neural network 18 has filtered the noise from the third filtered ultrasound image FI3, the invention can reduce the noise of the compound ultrasound image CI′ effectively, so as to improve the accuracy of the compound ultrasound image CI′. In this embodiment, the invention may use some manners including volting/average, neural network, alpha blending, multi-band blending, etc. to combine the first filtered ultrasound image FI1, the second filtered ultrasound image FI2 and the third filtered ultrasound image FI3 to form the compound ultrasound image CI′. However, the manner of combining images is not limited to the aforesaid embodiment.

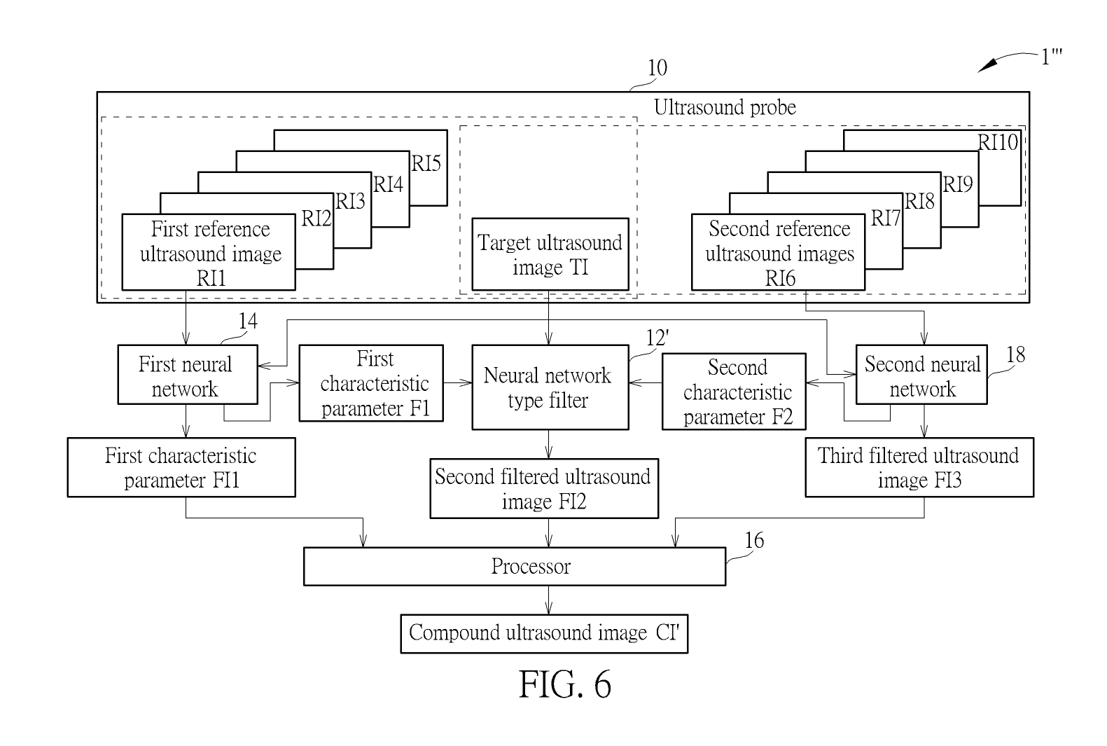

Referring to FIG. 6, FIG. 6 is a functional block diagram illustrating an ultrasound imaging system 1′″ according to another embodiment of the invention. The main difference between the ultrasound imaging system 1′″ and the aforesaid ultrasound imaging system 1″ is that the filter 12′ of the ultrasound imaging system 1′″ is a neural network type filter, as shown in FIG. 6. In this embodiment, the filter 12′ may also be a convolution neural network (CNN) or the like. In this embodiment, after generating the target ultrasound image TI and the first reference ultrasound images RI1-RI5, the first neural network 14 filters the target ultrasound image TI first according to the first reference ultrasound images RI1-RI5. Then, the first neural network 14 outputs a first characteristic parameter F1 to the filter 12′ after filtering the target ultrasound image TI. Furthermore, after generating the target ultrasound image TI and the second reference ultrasound images RI6-RI10, the second neural network 18 filters the target ultrasound image TI first according to the second reference ultrasound images RI6-RI10. Then, the second neural network 18 outputs a second characteristic parameter F2 to the filter 12′ after filtering the target ultrasound image TI.

Then, the filter 12′ filters the target ultrasound image TI according to the first characteristic parameter F1 and the second characteristic parameter F2 to generate the first filtered ultrasound image FI1. In this embodiment, the neural network type filter 12′ has been trained for filtering noise from the target ultrasound image TI according to the first characteristic parameter F1 generated by the first neural network 14 and the second characteristic parameter F2 generated by the second neural network 18. The invention may prepare a plurality of target ultrasound images TI in advance and the positions of the noises in the target ultrasound images TI have been known. Then, the target ultrasound images TI are inputted into the filter 12′ to train the filter 12′ to filter the noise from the target ultrasound image TI. It should be noted that the detailed training process of the neural network is well known by one skilled in the art, so it will not be depicted herein in detail. Furthermore, the manner of generating the characteristic parameter of the neural network is also well known by one skilled in the art, so it will not be depicted herein in detail. Since the first filtered ultrasound image FI1 is generated according to the first characteristic parameter F1 generated by the first neural network 14 and the second characteristic parameter F2 generated by the second neural network 18, the noise of the compound ultrasound image CI′ can be further reduced.

As mentioned in the above, after generating the target ultrasound image and the reference ultrasound images by different scanning parameters (e.g. scanning frequencies and/or scanning angles), the invention filters the target ultrasound image by the filter and filters the target ultrasound image by the neural network according to the reference ultrasound images, so as to generate the filtered ultrasound images. Then, the invention combines the filtered ultrasound images to form the compound ultrasound image. Since the filter has filtered noise from the filtered ultrasound image and the neural network has filtered noise from the filtered ultrasound image, the invention can reduce noise of the compound ultrasound image effectively, so as to improve the accuracy of the compound ultrasound image. Moreover, the filter may be a neural network type filter and filter the target ultrasound image according to the characteristic parameter generated by the neural network, so as to generate the filtered ultrasound image. Accordingly, the noise of the compound ultrasound image can be further reduced.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An ultrasound imaging system comprising:
an ultrasound probe generating a target ultrasound image and a plurality of first reference ultrasound images by a plurality of first scanning parameters;
a filter filtering the target ultrasound image to generate a first filtered ultrasound image;
a first neural network filtering the target ultrasound image according to the first reference ultrasound images to generate a second filtered ultrasound image; and
a processor combining the first filtered ultrasound image and the second filtered ultrasound image to form a compound ultrasound image.

2. The ultrasound imaging system of claim 1, wherein the first scanning parameter is a scanning frequency or a scanning angle.

3. The ultrasound imaging system of claim 1, wherein the filter is a neural network type filter, the first neural network outputs a first characteristic parameter to the filter after filtering the target ultrasound image, and the filter filters the target ultrasound image according to the first characteristic parameter to generate the first filtered ultrasound image.

4. The ultrasound imaging system of claim 1, wherein the ultrasound probe generates the target ultrasound image and a plurality of second reference ultrasound images by a plurality of second scanning parameters, the ultrasound imaging system further comprises a second neural network, the second neural network filters the target ultrasound image according to the second reference ultrasound images to generate a third filtered ultrasound image, the processor combines the first filtered ultrasound image, the second filtered ultrasound image and the third filtered ultrasound image to form the compound ultrasound image.

5. The ultrasound imaging system of claim 4, wherein the first scanning parameter is one of a scanning frequency and a scanning angle, and the second scanning parameter is another one of the scanning frequency and the scanning angle.

6. The ultrasound imaging system of claim 4, wherein the filter is a neural network type filter, the first neural network outputs a first characteristic parameter to the filter after filtering the target ultrasound image, the second neural network outputs a second characteristic parameter to the filter after filtering the target ultrasound image, and the filter filters the target ultrasound image according to the first characteristic parameter and the second characteristic parameter to generate the first filtered ultrasound image.

7. An ultrasound imaging method comprising steps of:
generating a target ultrasound image and a plurality of first reference ultrasound images by a plurality of first scanning parameters;
filtering the target ultrasound image by a filter to generate a first filtered ultrasound image;
filtering the target ultrasound image by a first neural network according to the first reference ultrasound images to generate a second filtered ultrasound image; and
combining the first filtered ultrasound image and the second filtered ultrasound image to form a compound ultrasound image.

8. The ultrasound imaging method of claim 7, wherein the first scanning parameter is a scanning frequency or a scanning angle.

9. The ultrasound imaging method of claim 7, wherein the filter is a neural network type filter, the first neural network outputs a first characteristic parameter to the filter after filtering the target ultrasound image, and the filter filters the target ultrasound image according to the first characteristic parameter to generate the first filtered ultrasound image.

10. The ultrasound imaging method of claim 7, further comprising steps of:
generating the target ultrasound image and a plurality of second reference ultrasound images by a plurality of second scanning parameters;
filtering the target ultrasound image by a second neural network according to the second reference ultrasound images to generate a third filtered ultrasound image; and
combining the first filtered ultrasound image, the second filtered ultrasound image and the third filtered ultrasound image to form the compound ultrasound image.

11. The ultrasound imaging method of claim 10, wherein the first scanning parameter is one of a scanning frequency and a scanning angle, and the second scanning parameter is another one of the scanning frequency and the scanning angle.

12. The ultrasound imaging method of claim 10, wherein the filter is a neural network type filter, the first neural network outputs a first characteristic parameter to the filter after filtering the target ultrasound image, the second neural network outputs a second characteristic parameter to the filter after filtering the target ultrasound image, and the filter filters the target ultrasound image according to the first characteristic parameter and the second characteristic parameter to generate the first filtered ultrasound image.
